(19) United States
(12) Patent Application Publication
(10) Pub. No.: US 2021/0369115 A1
(43) Pub. Date: Dec. 2, 2021

(54) TELEMETRY MONITORING AND ASSESSMENT SYSTEM OF PARAMETERS OF HUMAN VITAL FUNCTIONS AND CLINICAL SIGNS OF POSSIBLE FUNCTIONAL DISORDERS AND DETERMINATION OF METHODS OF CONTROL

(71) Applicant: Facecontrol, Inc., Miami, FL (US)

(72) Inventors: Alexander Zatvan, Miami, FL (US); Dmitri Kryjanovski, Miami, FL (US)

(73) Assignee: Facecontrol, Inc., Miami, FL (US)

(21) Appl. No.: 17/374,500

(22) Filed: Jul. 13, 2021

Related U.S. Application Data
(63) Continuation-in-part of application No. 16/632,504, filed on Jan. 20, 2020.

(30) Foreign Application Priority Data
Jul. 18, 2017 (EA) 201700326
Dec. 21, 2017 (EA) PCT/EA2017/000004

(51) Int. Cl. A61B 5/00; A61B 5/103; A61B 5/01; A61B 5/11; A61B 5/026; A61B 5/16

(52) U.S. Cl. CPC A61B 5/0022; A61B 5/1032; A61B 5/441; A61B 5/01; A61B 5/749; A61B 5/4845; A61B 5/026; A61B 5/7246; A61B 5/165; A61B 5/1124

(57) ABSTRACT

The software and interchangeable auxiliary hardware system for remote telemetric diagnostics of the physiological and mental state of a subject that includes an electronic computing device of a subject with a camera operably configured to measure spectroscopic changes, parameters of discoloration in the skin and the mucous membrane tissues in the areas of the subject's face, to provide assessment of pupils and sclera pigmentation, coupled with multiple interchangeable sensors to assess a surface temperature of the skin, composition of exhaled air from the subject, sensorimotor reaction of the subject, motor activity of the subject, and optical lighting operably configured to assess skin color and perfusion of blood vessels of the subject's face remotely; the electronic computing device of the subject communicatively coupled to a processor, operably configured through programed software instructions, to process the registered data and transmit it to a medical practitioner's personal computer to conclude with a diagnosis and/or recommendations.

### FIELD OF THE INVENTION

The proposed invention pertains generally to medicinal systems and methods, more specifically, the invention relates to a non-contact personal telemetric predictive automated diagnostic system and is intended for early detection of risks of functional disorders, states of intoxication, stress and emotional overload. This technology allows medical practitioners to conduct a comprehensive assessment of human health condition and to determine morphological changes in the subject human's body remotely by means of telemedical communication.

### BACKGROUND OF THE INVENTION

There are well-known and established virtual clinic telemedical methods and means of communication between a medical practitioner and a subject, that establish a connection within the framework of medical practice. Such framework uses known methods for managing medical records data in a telemedical system but with absolute inability to utilize conventional medical diagnostic equipment during remote medical consultation. In fact, such technical solutions do not allow for a comprehensive and effective medical assessment of the subject's condition when providing healthcare services remotely via telemedical session. Conventional diagnostic methods and equipment had been created for in-person medical appointments and are absolutely ineffective during telemedical sessions. The development of the telemetric biomedical technologies market is aimed at mastering the methodology of data mining to provide comprehensive reliable medical support, including predictive, variational models, building simulators and decision support systems for remote access. The capabilities of current microcirculation test methods in conjunction with an ability to obtain data from software and interchangeable auxiliary hardware system introduced herein, including a telemedical Internet based platform that provides access to large databases and knowledge, combining information, computing and measuring interchangeable auxiliary sensor components, containing an intellectual analysis system, an interactive consultation system, and a decision support system, allow non-tactile remote assessment of fundamental psychophysiological processes in the body including detection of the onset of functional disorders including temporary deviations caused by intoxication, mental and/or pscychoemotional state that were previously beyond reach.

Therefore, for the first time ever, the introduced invention closes a missing gap by allowing to provide the same reliable level of in-person medical assessment diagnostics during remote telemedical session between a subject and a medical practitioner.

### SUMMARY OF THE INVENTION

The invention provides a telemetry monitoring and comprehensive assessment of human health condition in subjects by evaluating system of parameters of human vital functions and clinical signs of possible functional disorders in subjects (employees, students, travelers, impaired subjects, persons under stress, etc.). This is achieved by means of the introduced non-tactile telemedical approach in conjunction with a proposed system of interchangeable auxiliary sensors and devices which allows to diagnose the overall physical condition of a subject and to detect the onset of functional disorders, including temporary deviations caused by drug or alcohol intoxication, and mental and/or pscychoemotional states. The invention also allows for the monitoring of the effectiveness of medical treatments and prescribed medicines. It can also provide prophylactic therapy and counseling remotely by means of a telemedical approach during a video conference calls or a pre-set automated scheduled medical examination with or without the participation of a medical practitioner.

Said another way, the present invention may be described as providing a software and interchangeable auxiliary hardware system for telemetric control of the physiological and mental state of a subject that includes an electronic computing device of a subject operably connectable with interchangeable auxiliary sensors, having an integrated camera, and having an audio input and an audio output, wherein the electronic computing device is operably configured to measure spectroscopic changes associated with the subject, parameters of color changes in facial skin and the mucous membranes tissues of the subject, conduct pupil assessment and a sclera discoloration analysis, measure surface temperature of skin of the subject, composition of exhaled air from the subject, and sensorimotor reaction of the subject, motor activity of the subject. Optical auxiliary lighting operably configured to assess skin color and perfusion of blood vessels of the subject's face and is communicatively coupled to a processor operably configured, through programed software instructions, to process the registered data received from the measurements obtained by the interchangeable auxiliary sensors to determine an overall health condition of the subject, including clinical signs of drug and alcohol intoxication that may impede general wellbeing of the subject, and to generate recommendations for a medical practitioner to consider via a wireless network with an electronic computing device of a medical practitioner communicatively coupled thereto.

This telemetry monitoring system, by using telemetric technologies of the software and an interchangeable auxiliary hardware system, also provides a method to determine and monitor clinical signs of a subject. These clinical signs would include the registration of changes in the color of the skin and mucous membranes, body temperature, vegetative-vascular reactions, mental activity, and disorders of the motor sphere. Moreover, assessment of the data obtained allows for analysis and submission of recommendations by the decision support system, while the registration of parameters is being carried out via a remote live feed interaction with a medical practitioner or pre-set automated scheduled medical examination with or without participation of a medical practitioner.

This technical result is achieved through the operation of the proposed invention, which consists of a telemetry monitoring system, that includes a camera and interchangeable auxiliary sensors for input, that monitors, reads and measures signals and parameters of a subject's vital signs. The invention does this by analyzing: acoustic, visual, pyrometric, spectroscopic data, skin surface temperature, sensorimotor reaction, motor activity, pscychoemotional and mental state analysis, pupil and sclera discoloration as well as the composition of exhaled air evaluation to determine clinical signs of drug and alcohol intoxication. Furthermore, the system operates in conjunction with optic devices utilizing computer vision technology to monitor parameters of facial skin and mucous membrane discoloration, on the basis of the asymmetry of microcirculation in blood vessels, using combined auxiliary lighting means to assess tissue pigmentation and perfusion of facial blood vessels, the detection and monitoring of the contraction of the facial expression muscles, monitoring and assessment of pupil and its movement, its reaction and size evaluation analysis. A connected computer processes the registered parameters, utilizing a computer software program designed to determine clinical signs, and to provide findings to a medical practitioner for evaluation.

The proposed invention enables independent assessment of vasomotor activity of resistive precapillary arterioles and precapillary sphincters, the exchange surface areas, and the efficiency of the filtration-reabsorption metabolic mechanism directly related to the microcirculation parameters and the exchange of water-soluble and low-molecular weight substances. The known method of applying predictive analytics includes the use of screening models for assessing the functional state of a subject. The current technological development in neurophysiology and bioelectronic medicine allows for a new look at the capabilities of test systems for assessing human adaptive and psychophysiological abilities.

A technical result to assess skin color and perfusion of facial blood vessels, as well as registering changes in the color of the facial skin and the mucous membranes is achieved by application of the provided method and telemetric technology of the software and interchangeable auxiliary hardware system that determines the primary physiological parameters of the subject by analyzing, evaluating, processing and recording the obtained data and results, which includes the projection of the subject's tissues, a change in the color gradient of the skin tissues and a vector change in monitoring points of the muscles of facial expression, wherein, the subject receives stimulation from visual, and/or audio, and/or other stimuli exposure for a set period of time depending on the nature of the assessment. All obtained data and a color synchronizing signal is received by a signal processing system and evaluated, taking into account set/known coefficients of change in color and coordinates to determine the required parameters.

With the foregoing and other objects in view, in accordance with the invention, a software and interchangeable auxiliary hardware system for telemetric medical assessment of the physiological and mental state of a subject is configured to measure spectroscopic changes associated with the subject's parameters of changes in facial skin and mucous tissues of the subject, skin's surface temperature, to provide pupil assessment and a sclera discoloration analysis as well as to evaluate a composition of exhaled air by the subject, to monitor a sensorimotor reaction and activity level of the subject. Its optical auxiliary lighting operably configured to assess skin color and perfusion of blood vessels of the subject's face. The electronic computing device of the subject is communicatively coupled to a processor operably configured, through programed software instructions, to process the registered parameters received from the measurements obtained from the interchangeable auxiliary sensors to determine immediate health condition of the subject by generating findings for a medical review through a computing device of the medical practitioner communicatively coupled to the processor.

In accordance with another feature, an embodiment of the present invention includes the computing device of the medical practitioner and a subject providing a graphic information input/output device operably configured to control a number of the registered parameters.

In accordance with yet another feature, an embodiment of the present invention also includes the computing device of the medical practitioner and a subject having a graphic information input/output device operably configured to determine a color gradient of image elements of the subject's tissues, in dynamics with a resolution of at least 30 frames per second.

In accordance with another exemplary feature, an embodiment of the present invention also includes the computing device of the medical practitioner having a user interface operably configured to view registered parameters including hemodynamic oscillations of the blood filling of the vessels of the subject's face.

In accordance with another feature, an embodiment of the present invention also includes a pyrometric infrared sensor operably configured to obtain a registered parameter that includes the temperature of the surface of the subject's facial skin and detecting correlation and blood perfusion oscillation registration parameters and surface temperature gradient.

In accordance with a further feature of the present invention, multiple interchangeable auxiliary sensors are operably configured to allow diagnostics of overall physical condition and early detection of the onset of functional disorders, including temporary deviations caused by drug or alcohol intoxication, mental and/or pscychoemotional state of the subject.

In accordance with an additional feature, an embodiment of the present invention also includes an audio information input device operably configured to register voice data and phoniatric changes of the subject during the consultation and examination.

Although the invention is illustrated and described herein as embodied in a telemetry monitoring and assessment system of parameters of human vital functions and clinical signs of possible functional disorders, and determination of methods of control, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “multiple,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time. Also, for purposes of description herein, the terms “upper”, “lower”, “left,” “rear,” “right,” “front,” “vertical,” “horizontal,” and derivatives thereof relate to the invention as oriented in the figures and is not to be construed as limiting any feature to be a particular orientation, as said orientation may be changed based on the user's perspective of the device. Furthermore, there is no intention to be bound by any expressed or implied theory presented in the preceding technical field, background, brief summary or the following detailed description.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. The terms “program”, “software program”, “software application”, and the like as used herein, are defined as a sequence of instructions designed for execution on a computer system. A “program”, “software program”, “computer program”, or “software application” may include a subroutine, a function, a procedure, an object method, an object implementation, an executable application, an applet, a servlet, a source code, an object code, a shared library/dynamic load library and/or other sequence of instructions designed for execution on a computer system.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a block diagram of an exemplary distributed data processing network using a personal mobile computing device or a personal computer of a subject, a personal computer device of a medical practitioner, and a server/database in connection with the application of the claimed invention all connected via Internet/WAN/LAN Network;

FIG. 2 is a block diagram of an illustrative electronic system, such as a personal mobile computing device, in accordance with the claimed invention;

FIG. 3 is a block diagram of the process of telemetric monitoring of parameters of the vital functions of the subject in accordance with the aspect of the claimed invention;

FIG. 4 is a screenshot of a software interface showing subject's facial area image during clustering and cardiointervalogram formation in accordance with the aspect of the claimed invention;

FIG. 5 is a block diagram of the possible location of a subject's relative position to a personal computing system in accordance with the aspect of the claimed invention;

FIG. 6 is a screenshot showing a set of the main waves when assessing and obtaining subject's vital functions in accordance with the aspect of the claimed invention;

FIG. 7 is a block diagram of a set of logical structures that implement various stages of the process, corresponding to the application of the claimed invention;

FIG. 8 is a diagram of the coordinate distribution of control points, the vector displacement of which is one of the diagnostic features in accordance with the aspect of the claimed invention;

FIG. 9 is a screenshot illustrating the possibility of estimating the parameters of the pulse wave by monitoring the nail phalanx of a finger in accordance with the aspect of the claimed invention;

FIG. 10 is a block diagram of a multifunctional telemetry monitoring and comprehensive assessment Internet platform to provide remote medical examinations of human health condition to subjects by means of non-tactile telemedical approach in conjunction with proposed system of interchangeable auxiliary sensors utilizing subject's personal computing device, personal computer of a medical practitioner, a server/database all connected via Internet/WAN/LAN Network in connection with the application of the present invention;

FIG. 11 is another block diagram of a multifunctional corporate telemedicine Internet platform for remote medical examination using subject's computing mobile device integrated with interchangeable auxiliary sensors and a server/database all connected via Internet/WAN/LAN Network in connection with the application of the present invention;

FIG. 12 is a block diagram of a hardware and software system for telemetry monitoring of vital parameters of a subject and decision-making by medical practitioner when conducting a remote medical examination in accordance with an aspect of the present invention;

FIG. 13 is a diagram of a recommended body positioning for a subject relative to a personal computing device and an interchangeable auxiliary hardware in accordance with an aspect of the present invention; and

FIG. 14 is a diagram illustrating an example of an assessment process of clinical signs of drug and/or alcohol intoxication.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms depending on set objectives and purposes of application.

The present invention provides a novel and efficient telemetry monitoring and assessment system of parameters of human vital functions and clinical signs of possible functional disorders by means of introduced non-tactile telemedical approach in conjunction with proposed system of interchangeable auxiliary sensors and devices allowing remote diagnostics of overall physical condition and early detection of the onset of functional disorders, including temporary deviations caused by drug or alcohol intoxication, mental and/or pscychoemotional state;

The proposed invention pertains to telemedicine, more specifically, the invention relates to non-tactile telemetric personified predictive automated monitoring diagnostic system intended for early detection and monitoring of health condition and risks of functional disorders, correction of psychosomatic disorders, and morphological changes in the human body, including states of intoxication, stress and emotional overload remotely, utilizing the system of telemedical examinations during a video conferences call or pre-set automated scheduled medical examination with or without participation of a medical practitioner.

The capabilities of current microcirculation test methods in conjunction with an ability to obtain data from a software and interchangeable auxiliary hardware system introduced herein, including a telemedical Internet platform that provides access to large databases and knowledge, combining information, computing and interchangeable auxiliary measuring sensor components, an intellectual analysis system, an interactive consultation system, and an decision support system, that allow non-tactile remote assessment of fundamental physical and psychological processes that were previously beyond reach.

The development of the biomedical telemetry market is aimed at mastering methods of intellectual data analysis, including predictive variable models, simulators, and decision-making support systems.

The proposed invention enables to assess the vasomotor activity of resistive precapillary arterioles and precapillary sphincters, the exchange surface area, the efficiency of the filtration-reabsorption metabolic mechanism directly related to the microcirculation parameters and an exchange of water-soluble and low-molecular weight substances. The known method of applying predictive analytics includes the use of screening models for assessing the functional state. The current technological development in neurophysiology and bioelectronic medicine enables a new look at the capabilities of test systems for remote assessment of human adaptive and psychophysiological states.

The aforesaid technical effect is achieved by a telemetry control system of subject's vital functions assessment system devices for measuring, input and reading of signals, parameters, and its graphic data analysis, including coordination of devices based on computer vision technologies that allow monitoring of asymmetric tissues color change parameters in subject's facial and mucous tissues, a processor which is connected to the devices for processing the recorded parameters, and a device to display graphic data of the subject's image. The processor is configured to process the input signals based on the preset color change coefficients to determine the optical density of tissues, plethysmography data, and oxygenation parameters.

In addition, the telemedical Internet platform includes a subject's interface, an automated workstation for remote access, a software and interchangeable auxiliary hardware system for telemetric control of physiological functions and psychological, including emotional state of a subject, connected by Internet/WAN/LAN Network communication channels with the server part.

Additionally, the subject's interface of the platform may include an automated workstation of a subject allowing a remote access of a medical practitioner, consisting of a telemetric monitoring system via Internet channels providing an ability to receive data from the subject's software and interchangeable auxiliary hardware system to determine subject's immediate state of health, including but not limited to possibility of intoxication and/or physical and/or emotional exhaustion.

A server part of the platform may include and/or contain a system to determine access rights to the subject's interface, identification and recognition system of a subject, providing access to databases and knowledge, combining information and computational components, a data mining system, an interactive consultation system and a support system providing results and recommendations.

The technical effect achieved through the use of the proposed invention provides a non-tactile comprehensive assessment of fundamental physiological and psychological processes of human health in subjects (for instance: employees, students, travelers, impaired subjects, persons under stress, operators of heavy machinery and/or commercial transportation, etc.) by means of introduced remote telemedical approach allowing early detection and monitoring of the onset of functional disorder risks including temporary deviations caused by intoxication, mental and/or pscychoemotional state, ineffectiveness of medical treatment and prescribed medicine during a live feed by means of telemedical approach during a video conference or pre-set automated scheduled medical examination with or without participation of a medical practitioner based on facial microcirculation asymmetry, facial muscle contractions and receipt of additional data from software and interchangeable auxiliary hardware system which is an integral part of a multifunctional system of a telemedical Internet platform that provides access to large databases and knowledge, combining information and computing components and devices, containing an intellectual analysis system, an interactive consultation system, and an decision support system.

The technical results of the present invention are achieved by operating the proposed telemetry monitoring system that includes or consists of devices for input of graphic visual and sound data, processor, server, personal computer, a software and in addition to having, containing, measuring interchangeable auxiliary sensor devices for input and reading of signals and parameters of a subject's vital signs and data of graphic, acoustic, text, pyrometric, spectroscopic information and coordinate devices using computer vision technology to monitor the parameters of other conditions and reactions, including skin and mucous tissues' color alteration on the basis of the asymmetry of microcirculation and perfusion of the blood vessels and the contraction of the muscles of facial expression using combined light sources of various technical properties. Furthermore, interchangeable auxiliary measuring sensors and devices allow the system to analyze a temperature of a skin surface of the subject, pupil tracking and evaluation as well as the composition of exhaled air to determine clinical signs of alcohol and drug intoxication, both sensorimotor and vegetative-vascular reactions, motor activity. A system processor is connected to registered parameters, containing a computer software program that provides assessment of the data obtained, its analysis and submission of results and potential recommendations in the decision support system, while the registration of parameters is carried out during a live feed of remote video interaction with a medical practitioner or pre-set automated scheduled medical examination with or without participation of a medical practitioner over an electronic computing device. The system also allows to conduct scheduled routine—a pre-set medical and general examinations, with or without live participation of a medical practitioner. In case of examination without a medical practitioner, the result of an examination is automatically stored on the server for a later review. Moreover, an analytical system may be programmed to provide its findings of the fact of intoxication of a subject without a need to involve a medical practitioner in the process to authorized parties automatically.

Evaluation and analysis of primary physiological parameters of a subject, using the graphic information input device, the processor, personal computer, server and a software to register changes in the color of the skin and mucous membrane tissues, to assess skin discoloration and perfusion of blood vessels of the facial tissues are determined by recording, on projections of the tissues of a subject's face, a change in the color gradient of the tissues and a vector change in monitoring points of the muscles of facial expression, wherein the subject looks at a predetermined image or set of images and/or any type of a graphical data by means of display or other stimuli for a set period of time depending on the nature of the assessment. All data obtained during the evaluation, including the color of elements of analyzed tissues and also a color synchronizing signal are received by a signal processing system, and the signals are evaluated considering set or known coefficients of change in color and coordinates for determining the required parameters.

The device for input and reading of signals, parameters, and graphic data of parameters is made with the possibility to monitor the tissue color gradient with a frequency of at least 30 frames per second.

The device for input of graphic data with the processor connected to it is constructed to allow the use of computer vision to coordinate distribution of control points and the vector displacement of facial areas and facial contractions.

During testing, a subject may be placed at a distance of approximately no more than three feet 1 m) from the input computing device in a sitting position with legs not bent at the knees in a state of relative rest for a predetermined time. The obtained data of tissues' discoloration, as well as the color synchronization signal, enter the signal/data processing software program, and the signals with all data are evaluated taking into account the specified or known color change coefficients to determine the required parameters: heart rate, respiration rate, oscillation amplitude of slow physiological waves of the subject: pulse waves, respiratory waves, waves caused by parasympathetic or sympathetic cholinergic influences, waves caused by the intrinsic myogenic activity of myocytes of micro vessels, waves caused by the influence of sensory peptidergic nerve fibers on neuropeptides myocytes, waves caused by low-frequency rhythm of impulses of sympathetic adrenergic waves of vasomotor fibers caused by the influence of endothelial nitric oxide.

The claimed method for telemetric monitoring of subject's vital functions using the system, includes detection and assessment of the basic psychological parameters and evaluation of the obtained data. These parameters are determined using the graphic information input device and the processor by tracking and recording changes in the color chromaticity gradient and vector changes at the control points of the facial muscles using projections of the subject's face; at the same time, the subject looks at a predetermined image or set of images and/or any type of a graphical data by means of display or other stimuli for a set period of time, after which the obtained data of the color of the elements of analyzed tissues and also a color synchronizing signal, enter the signal processing system, and the system evaluates the signals taking into account the specified/known coefficients of changes in the chromaticity and coordinates to determine the required parameters. The system records the signals of changes of the color of the images of the tissue elements and vector changes in the coordinates of the control points in at least 2 areas of the subject's facial skin at preferably twelve control points, at the same time, wherein the subject looks at a predetermined image or set of images and/or any type of a graphical data by means of display of the computing device designed to display graphic information or other stimuli for a for a predetermined time.

The data received from the input device and processed by the processor's software program allows to determine the amplitude parameters of the oscillations of slow physiological waves of a human, with high accuracy: pulse waves, respiratory waves, as well as waves that are caused by parasympathetic or sympathetic cholinergic influences, the myogenic activity of microvascular myocytes, the influence of sensory peptidergic nerve fibers on neuropeptide myocytes, the low-frequency rhythm of impulses of sympathetic adrenergic vasomotor fibers, and the effects of endothelial nitric oxide. Recording of signals related to changes in the color of the elements of the facial tissues' images of the subject and vector changes in the coordinates at control points from the projection of the subject's face makes it possible to determine blood perfusion in areas as small as 0.31 sq inches (2 sq cm). In addition, recording of parameters related to changes in the color of image elements and vector changes in the coordinates at control points from the projection of the subject's face allows to identify the asymmetry of parameters: amplitudes of oscillations of slow physiological waves and manifestations of emotionally conscious and unconscious facial micro mimic expressions that may be used by the system of telemetric detection to evaluate transmitted information.

The claimed invention is a system and a method for early detection of risks of functional disorders, assistance in correction of psychosomatic disorders, and morphological changes in the human body, and monitoring of the efficiency of preventive measures considering the parameters of the amplitude of oscillations of physiological slow waves, the asymmetry of microcirculation of blood through the vessels, as well as conscious and unconscious facial muscle contractions.

Furthermore, the device for input/output of graphic information allows to determine the parameters of the color gradient of the image elements of the tissues, in dynamics with a resolution of at least 30 frames per second, and to evaluate the parameters of thermodynamic oscillations of the blood filling of the vessels of the face. In this case, the subject placed at a distance of no more than approximately 3 feet 1 m) from the input/output device in a sitting position with legs not bent at the knees in a state of relative rest for a predetermined time. The obtained data of the color of the image elements, as well as the color synchronization signal, enters the signal processing program, and the signals are evaluated taking into account the specified/known color change coefficients to determine the required parameters: heart rate, respiration rate, oscillation amplitude of slow physiological waves of the subject: pulse waves, respiratory waves, waves caused by parasympathetic or sympathetic cholinergic influences, waves caused by the intrinsic myogenic activity of myocytes of micro vessels, waves caused by the influence of sensory peptidergic nerve fibers on neuropeptides myocytes, waves caused by low-frequency rhythm of impulses of sympathetic adrenergic waves of vasomotor fibers caused by the influence of endothelial nitric oxide.

The method of telemetric control of the parameters of the vital functions of the subject using a software and interchangeable auxiliary hardware system allows the registration of the dynamics of changes in the subject's tissues by splitting image elements into frames, extracting facial tissue elements, filtering according to the determined condition of a natural skin and mucous membrane tissue color, evaluating the change in the color gradient of each pixel of the frame of subject's tissues, calculating values for the channels of the additive RGB color model, taking into account the specified coefficients of color change, to determine the optical density of tissues, hemodynamic parameters of pulse waves, calculating heart rate and heart rate variability. The obtained data can be processed directly by the processor or sent via the Internet network to a server for signal processing, upon which the signals are evaluated considering the requirements set by a medical practitioner, or by an algorithm previously defined for a particular subject, utilizing established conventional medical standard techniques.

On the basis of the above visual image processing algorithm, the present invention makes it possible to estimate the parameters of the oscillation amplitude of slow physiological waves of a person, such as pulse waves, respiratory waves, waves caused by parasympathetic or sympathetic cholinergic influences, waves caused by their own myogenic activity of micro vessel myocytes, waves caused by the influence on myocytes of neuropeptides of sensory peptidergic nerve fibers, waves caused by low-frequency rhythm of impulses of sympathetic adrenergic vasomotor fibers, waves caused by the influence of endothelial nitric oxide, and to assess the state of the vegetative-vascular system of the body remotely from the subject.

The method can be used to assess changes in vegetative-vascular reactions, taking in consideration the temperature characteristics of the surface of the tissue and the lighting conditions of the premises where a medical examination takes place, synchronous registration of changes in facial tissue color and temperature from its surface area allowing to assess hemodynamic parameters (heart rate, blood pressure, features of local perfusion).

The proposed method includes a qualitative and quantitative assessment of changes in the color of the facial tissue and the surface of mucous membrane tissues (tongue, oral mucosa, sclera of the eyes), for which, in real time, from the video stream, a computer software program using computer vision technologies selects the necessary investigated elements of the facial image or parts of it and performs numerical and graphic processing of color indicators based on intellectual and statistical analysis of the data obtained, automatically identifies signs (injections, hyperemia, pallor) and generates its outcome and results in form of recommendation.

The method includes registration of facial image elements and vector control of changes in projections of points to assess the contraction of the head muscles innervated by the somatic nervous system (cranial nerves) to assess eye-motor reactions (eyeball, pupil, eyelids), allowing to identify clinical signs of disorders (expansion, narrowing, sluggish reaction to light, unresponsiveness).

The method for determining clinical signs provides for the possibility of detecting and assessing speech disorders (dysarthria, aphasia, dyslexia) during the survey, the phoniatric assessment of acoustic information is carried out by the computer software program algorithm based on the analysis of the features of the voice timbre, the duration of pauses when answering questions, loudness, duration (stretch), the presence of hesitations in order to determine objective quantitative indicators that are compared with the reference ones, which allows the decision support system to draw medical conclusions and provide recommendations to a medical professional.

The claimed method makes it possible to use in the process of medical examinations test systems for quantitative assessment of selectivity and concentration of attention, emotional and cognitive control, modified Eriksen flanker tasks used in the program, recognized by the American Board of Psychiatry and Neurology and the American Board of Preventive Medicine, allowing high accuracy in identifying mental disorders, physical activity deviation, the degree of inadequacy (intoxication), the algorithm of evaluation allows to determine all initial signs, processes in by a subsequent decision support system to provide recommendations on the subject's condition.

The audio information input device is configured to register voice data and phoniatric changes during the medical examination process.

Combined auxiliary optical illumination devices with a color temperature 2800 to 3800 K are intended to provide enhanced assessment of blood perfusion of superficial and deeper vessels of the face with programmable adjustments of illumination parameters.

The pyrometric infrared sensor is designed to register the parameters of the surface temperature of the facial tissues and detect the correlation, the parameters of the oscillation of blood perfusion and the temperature gradient of the surface temperature.

Spectrophotometric infrared sensors are made with a wavelength range of 3 to 9 microns and/or electrochemical sensors are designed to monitor ethanol vapors in exhaled air.

The coordinating processing device allows to perform tasks related to the assessment of sensorimotor reactions and pscychoemotional state in the process of research.

Processing of the received signals, according to the algorithms provided in the software program, identifies the subject, evaluates the basic physiological parameters, namely, pulse rate, respiratory rate, blood pressure, body surface temperature, characteristics of the color parameters of the mucous tissue surfaces of the tongue, oral mucosa and sclera of the eyes, reveals phoniatric, sensorimotor and motor disorders. The system includes a computer software program that allows to automatically provide recommendations and/or assist a medical practitioner in a decision-making process when evaluating subject's physical and psychological states, analyzing changes in vegetative-vascular reactions, disorders of the subject's motor sphere and their mental capacity, evidence of intoxication.

The software and interchangeable auxiliary hardware system include an audio input device that allows recording and evaluating the acoustic characteristics of the subject's voice data in the frequency range up to 18 kHz based on spectral analysis, the main properties of sound are subject to control: frequency (height), strength (amplitude), intensity, etc. the duration, as well as the duration of the pause and the presence of hesitations. In this case, the registration of the subject's voice data is carried out during the interview with the simultaneous registration of the visual image of the subject. The received data from the device is sent to the signal processing software program, while the analysis is carried out considering the given (known) characteristics of the subject's voice in order to detect the magnitude of phoniatric changes (dysarthria, dyslexia, aphasia, etc.), to identify clinical signs that impede the performance of functional duties, as well as the possibility of additional authentication.

The software and interchangeable auxiliary hardware system include combined optical illuminating lighting devices, including a set of point light sources with a spectrophotometric temperature of 2800, 3200, 3800, 4300 K, located so that the optical axes of the light sources forming a separate group are parallel to each other and directed towards the subject. The characteristics of point light sources in terms of color temperature ensure the objectivity of color reflection and the reliability of registration of blood perfusion of superficial and deeper vessels of the facial tissues.

The software and interchangeable auxiliary hardware system include an infrared sensor module designed for high-precision non-contact temperature measurements of the surface of the subject's facial skin, while the distance between the sensitive element of the pyrometer and the skin surface should not exceed 3 feet 1 m). The combined use of the module in conjunction with a graphic information input device and optical means of illumination makes it possible to reveal the correlation of the parameters of the oscillations of blood perfusion and the temperature gradient of the surface temperature, regardless of the ambient temperature.

The software and interchangeable auxiliary hardware system include a camera that allows tracking and recording to evaluate subject's pupil's reaction to external stimuli, for example, to forms of light combinations generated by illuminating lighting devices from an interchangeable auxiliary sensor hardware system, as well as analysis of discoloration of sclera of the subject's eyes to detect signs of a drug intoxication and/or other clinical signs.

The software and interchangeable auxiliary hardware system include spectrophotometric infrared sensors with a wavelength range of 3 to 9 microns and/or electrochemical sensors designed to monitor ethanol vapors in exhaled air, exceeding the values of the norms determine by the State law, in order to detect intoxication or residual signs of alcohol consumption.

The software and interchangeable auxiliary hardware system include a coordinate input device that allows, in the process of analysis, to perform functions, related to an assessment of sensorimotor reactions and evaluation of a pscychoemotional state, for example, the implementation of Eriksen flanker task for the selectivity and concentration of the subject.

The system contains a mathematical algorithmic computational software that allows processing of the received data considering the embedded algorithms for subject identification, assessing the main physiological parameters (pulse rate, respiratory rate, blood pressure, body surface temperature), characteristics of the color parameters of the mucous tissue surfaces of the tongue, oral mucosa, pupils and sclera of eyes, to identify phoniatric, sensorimotor and motor disorders, as well as signs of an intoxication caused by drugs and/or alcohol.

The computer software program (system of support for making recommendations for a medical practitioner) allows to automatically provide recommendations and carry out suggestions according to established conventional medical practices regarding determined changes and vegetative-vascular reactions, disorders of the motor sphere, and in mental activity which effects general wellbeing of a subject.

The invention solves a problem that was previously out of reach to allow for a medical practitioner to provide a timely and highly effective medical assessment examination remotely via telemedical session using said software and non-tactile interchangeable auxiliary hardware system without the physical presence of medical practitioner in the place of examination and to be able to receive all necessary diagnostics information at the same level as conventional medical devices bring during a personal medical examination which incomparably, for the first time ever, increases the efficiency of the process of remote telemedical examination taking it to an absolute new level.

Considering the above and other considerations and in accordance with the claimed invention, the present artwork also provides a graphic information describing the proposed invention. The following graphic information provides a sample component list of parts comprising claimed invention with intent to describe a founding method and a sample capability of the system. The system parts, including processors, servers, computers, mobile gadgets, sensors, emitters, devices, and software, may all be altered, removed, substitutes, upgraded based on a set goals and pursued types of information to be discovered; provided graphical information includes description of an input/output device, a processor for implementing a method for detection of deviations of received health data when diagnosing a subject. The method involves using a computer software program for command execution stored in memory using at least one processor of an electronic device to receive data describing human heart rate variability while simultaneously to record and to process the elements of the analyzed image and to track changes in the color of facial tissues, based on the specified algorithms.

The method further provides the possibility of recording subject's face for a predetermined period of time using the digital camera of the electronic device or of the interchangeable auxiliary hardware system. Images and videos are processed to track changes in the tissue color gradient and to monitor the subject's facial muscle contractions.

Furthermore, the method includes ability to record a video of a subject by utilizing a digital camera with a resolution of at least 640×480 pixels, dividing the image elements into individual frames, determining the elements of the face, filtering the tissue color according to the specified conditions, coordinating distribution of the boundaries of areas that are subject to investigation, calculating averages for the channels of the additive RGB color model, noise filtering, and drawing a cardiointervalogram. A digital camera may be a part of a personal computing device or may be integrated as part of an interchangeable auxiliary hardware system.

The claimed system includes a monitoring module to analyze conscious and subconscious facial muscle contractions, thus allowing to evaluate the physiological mechanisms of the individual stages of the information processing: sensory analysis, activation of attention, retrieving of memory standards, favorable decision-making process, etc.

The set of computer commands includes commands to turn on the graphic information input/output device and record image parameters, including coordination of the device that utilizes computer vision technology to record a video of an image of a person's face for a specified period of time. Then, the recorded video is processed to assess the parameters of vital functions and create recommendations to a medical practitioner for consideration.

The system uses a processor with a software-mathematical computational algorithm to determine the main waves in the processed signals to control the subject's vital activity and provides recommendations to a medical practitioner with suggestions of treatment based on established conventional medical practice.

The method for telemetric monitoring of subject's vital functions using the claimed system includes determination of the basic physiological parameters, assessing, and analyzing the data, as well as recommending treatment based on established conventional medical practice. In this case, the parameters are determined using the image input/output device and the processor by recording changes of the color of the subject's facial tissues and facial muscle contractions, wherein the subject looks at a predetermined image or set of images and/or any type of a graphical data by means of display or other stimuli for a set period of time. The obtained data can then be processed directly by the processor or transmitted via the Internet/WAN/LAN network to the server for data processing, the data is evaluated according to the requirements set by the medical practitioner, otherwise the predetermined algorithms for a particular subject or standard methods are used.

The claimed method can also be implemented as a mobile application for the telemetric monitoring system that allows to evaluate the received diagnostics analytics obtained via remote Internet platform system, compare it with subject's previously obtained data, and provide recommendations utilizing support model algorithms to a medical practitioner when assessing subject's health condition.

To evaluate subject's psycho-emotional state, a software program allows to generate external stimuli in form of visual graphics and evaluate subject's reactions to it. An assessment may be conducted during an interview, psychological testing, a game to evaluate the sensory-motor response; all actions and reactions of a subject are automatically monitored; the system tracks physiological parameters such as pulse, respiration, second-order waves from the description above.

Generating external stimuli and tracking of the facial micro expressions, including asymmetry of micro expressions of a subject in the process of recording parameters using the graphic information input/output device and analyzing it using a software program, taking into account at least nine control points, allows to evaluate the physiological mechanisms at the individual stages of the information processing: sensory analysis, activation of attention, formation of images, retrieving of memory standards, decision making, etc.

The claimed method allows to monitor a micro mimic contractions of facial muscles controlled by the somatic nervous system (cranial nerves); these processes involve sensory and motor nerves of the muscles and skin that belong to the parasympathetic nervous system. The parasympathetic nervous system also includes the facial nerve (facial muscles) and the ocular motor nerve (eyeballs and eyelids).

In order to reliably estimate facial muscle contractions by vector analysis, the coordinates of the control points of the face image must be recorded at intervals of less than 0.05 seconds.

The graphic information input device records color gradient changes, i.e., each pixel of the frame of the facial tissues color alterations when the pulse wave propagates.

The graphic information input device can be used to determine the mobility (lability) of muscular and nerve tissues in the presence of a source of liminal irritation in the form of light (color) stimulation. For example, to assess the lability of vegetative fibers, the software program uses the graphic information input system and generates a red rhythmic stimulus of maximum brightness with a frequency of 200 pulses per second by a source of liminal irritation in the form of light (color) stimulation.

Optional synchronized assessments of the microcirculation in blood vessels, the micro mimic processes of pain sensitivity control, and the effects of cold allow to determine the proportion of participation of each action and calculate the correlation between emotional and psycho-physiological reactions. The software program also includes a method for assessing the correlation between data from various actions which allows to distinguish between individual spontaneous unconscious emotionally significant micro expressions and feelings that are expressed by facial muscles.

A requirement to evaluate at least two areas of the facial skin tissue is determined by the microcirculation system. The microcirculatory vasculature that comprises repetitive functional units, modules, each of which is a specific multicomponent system consists of nerve conductors, organ cells, and micro vessels (arterioles, precapillary arterioles, capillaries, postcapillary venules, collecting and other venules, arteriole-venular anastomoses, and lymphatic vessels).

This system of functional structures ensures the maintenance of homeostasis and blood-lymphatic balance. Each microcirculatory module is separated from the neighboring ones both structurally and functionally since it has isolated pathways of inflow and outflow of blood and tissue metabolism products.

Algorithms of mathematical analysis implemented in this system are used to compare the microcirculatory modules of facial projections and provide a significant diagnostic feature.

The graphic information input device and a processor allows to evaluate changes in muscle activity, neural signals, and rheography parameters of blood when recording afferent (sensory) and efferent (motor) reactions in the range of physiological stimuli of different directions that are stimulated by using a graphic information display system. Evaluation of the recorded data allows to determine exactly which graphic images caused subject's subconscious reaction. Based on this information, the system generates results and displays these exact graphic images that caused subject's subconscious reaction. Areas of application of this specific functionality is vast as it allows to learn about personal, true preferences of subjects in different areas of everyday life, particularly when performing a drug addiction assessment in subjects trying to hide the fact of addiction.

Thus, the method of assessing the state of the mechanisms of regulation of physiological functions in a human body, in particular, the general activity of regulatory mechanisms, neuro-humoral regulation of the heart, relationships between the sympathetic and parasympathetic parts of the autonomic nervous system, involves the use of standard statistical methods and algorithms of mathematical analysis. To assess the reliability of the said method, the obtained data was analyzed and compared to data obtained from conventional diagnostic methods.

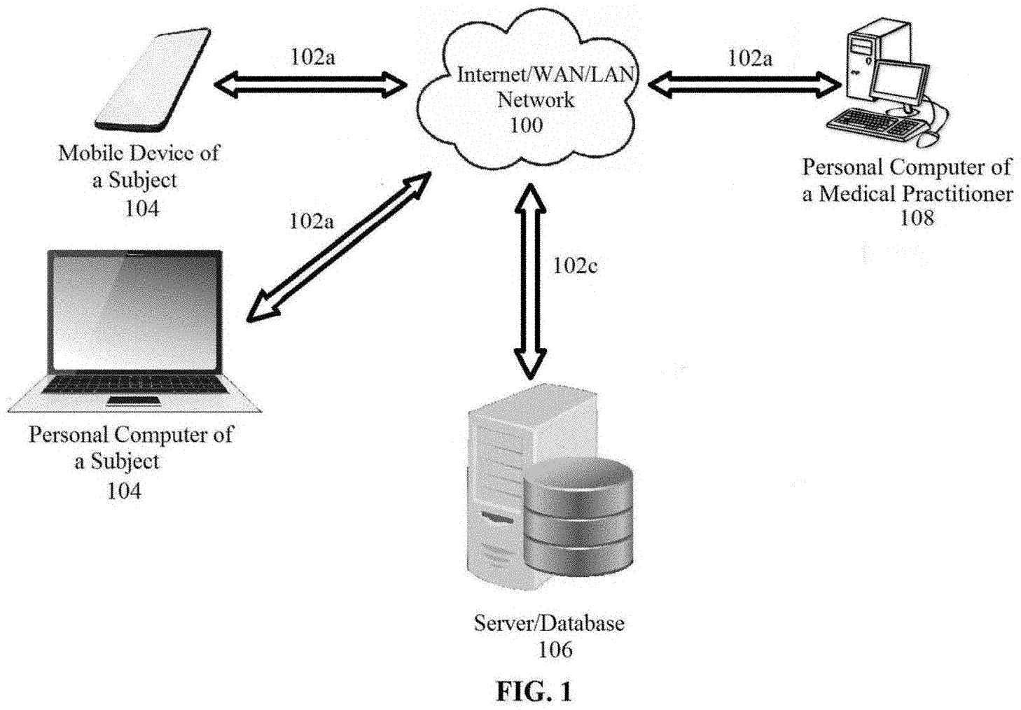

Furthermore, with reference to FIGS. 1-9, embodiments of the invention are shown. Specifically, FIG. 1 depicts a block diagram illustrating an exemplary network of a data processing system in which the claimed invention may be implemented. FIG. 1 illustrates some of the advantages of the claimed invention, however, as described below, the invention itself can be embodied in several shapes and sizes with different combinations of properties and elements and different numbers of components and their functions. The first example, as shown in FIG. 1, includes connections 102a-c, which are the medium for communication between various devices and computers interconnected in Internet/WAN/LAN 100. Connections 102a-c may be wired or wireless. Examples of wired connections are Cable, telephone line, and fiber optic cable. Examples of wireless connections are Internet, Radio frequency (RF) and infrared (IR), Wi-Fi, Bluetooth. Many other wired and wireless connections are known in this technical field and can be used concurrently with the claimed invention.

In this example, the Internet/WAN/LAN 100 includes an electronic device, such as subject's personal computer or mobile computing device 104, a server 106, and a personal computer of a medical practitioner 108. Subject's personal computer or mobile computing device 104 may be used to execute programming commands contained in software that can be obtained from server 106 via Internet/WAN/LAN 100. In other embodiments of the invention, a personal computer of a medical practitioner 108 may execute programming commands received from the server 106 over the Internet/WAN/LAN 100. In other embodiments, the software is a web application, desktop software, or a mobile application. In one embodiment, the network is an Internet. The Internet is a worldwide collection of networks and gateways that use the TCP/IP protocol suite to communicate with each other. The Internet is based on a unifying backbone consisting of high-speed data lines between the main nodes or host computers represented by thousands of commercial, government, educational, and other computer systems that route data and messages. Of course, Internet/WAN/LAN 100 may also be implemented as many different types of networks, such as, for example, an intranet, a LAN, or a cellular network. FIG. 1 is considered as an example rather than a structural limitation for the claimed invention.

The server 106 can be considered as a computer that controls access to the centralized resources or databases. In some embodiments of the invention, subjects undergoing assessment using a personal mobile or computing device 104 may request the software, which is an example of the use of the claimed invention. The server 106 can receive, process, and execute requests by transmitting the software to the personal computer and/or mobile computing device 104 via Internet/WAN/LAN 100. In other embodiments, a personal computer of a medical practitioner 108 may request the software and/or other data, and the server 106 may receive, process, and execute the request by transmitting the software/data to the personal computer of a medical practitioner 108 via Internet/WAN/LAN 100.

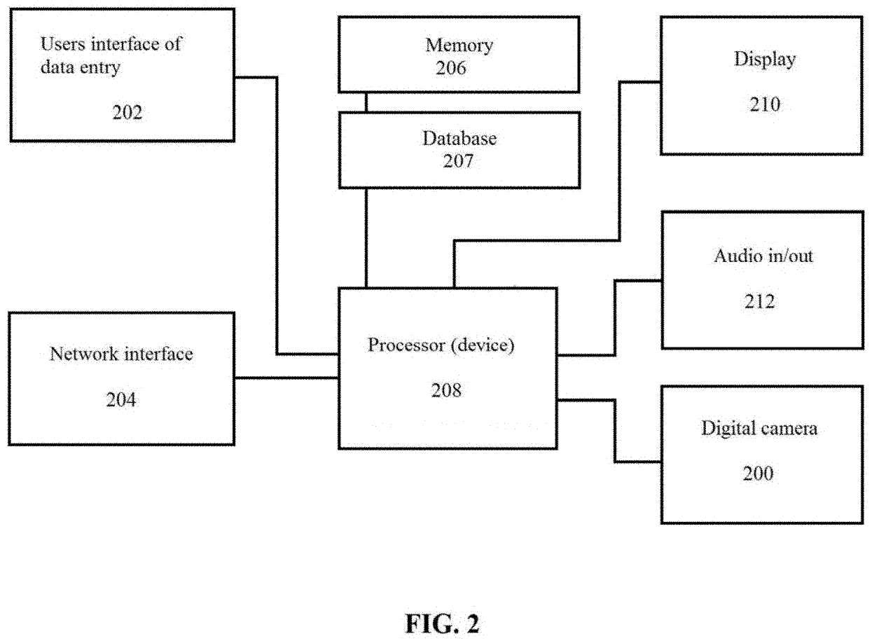

With reference to FIG. 2, a subject's personal computer and mobile computing device as well as medical practitioner's personal computer 104 and 108 (provided in FIG. 1) is illustrated in the block diagram. A personal computer, a mobile computing device 104 and 108 have the following components: camera 200, user input interface 202, network interface 204, memory 206, processor 208, a display 210, audio input/output 212.

The camera 200 can be used to record still and video images. The camera 200 is a digital camera that allows to store images in memory 206 that are processed by the processing device 208. The camera 200 is connected with a microphone that records sound, including simultaneously with captured images of a subject. The camera 200 is preferably used to record images with a resolution of at least 640×480 pixels to allow accurate interpretation and analysis of images in accordance with the methods described herein, as well as methods well known in this technical field. Lower quality cameras may not capture high-resolution images. The user input interface 202 provides subject and medical practitioner to input data into a personal computer or mobile computing device 104 and 108 correspondingly. The user input interface 202 may also facilitate interaction between users of 104 and 108 devices.

The user input interface 202 is a keyboard that supports a variety of user input operations. For example, the keyboard may include alphanumeric keys for entering (for example, contact information, text, etc.). The user input interface 202 may include special function keys (for example, to release the camera shutter and adjust the volume, the Back button, the Return button, etc.), as well as navigation and selection keys, the cursor, and so on. Keys, buttons, and/or keyboards may be implemented as a touch screen associated with the display 210 of a type known in this technical field. The touch screen can also provide data output or feedback to the user, for example, feedback or adjusting the keyboard orientation in accordance with the signals of motion sensors, such as an accelerometer located inside subject's mobile computing device 104.

The network interfaces 204 may include one or more network interface cards or a network controller. In some embodiments, the network interface 204 may include PAN, a personal area network interface. The PAN interface may allow the personal computer and a mobile computing device 104 to connect to the network using a short-range data transfer protocol, such as Bluetooth. The PAN interface allows one personal computer 104, 108 to establish a wireless connection with another personal computer or a mobile computing device 104 using a peer-to-peer connection.

Network interfaces 204 may also include LAN, a local area network interface. The LAN interface can be, for example, a wireless LAN interface, including Wi-Fi. The range of the LAN interface usually exceeds the range available for the PAN interface. In most cases, a connection between two electronic system via a LAN interface may include a network router or other intermediate system.

In addition, network interfaces 204 may include connections to an Internet—a global WAN through a global WAN interface. The WAN interface can provide connectivity, for example, via cellular networks. A WAN interface can include elements such as an antenna connected to a radio circuit having a transceiver system for transmitting and receiving radio signals using the antenna. The radio circuit can be configured to operate in a cellular network, including, but not limited to, GSM global mobile communications systems, CDMA code division multiple access, wideband CDMA, etc.

A personal computer or mobile computing device 104 may also include an NFC short-range communication interface. The NFC interface can provide extremely close communication range at relatively low data transfer rates (for example, 424 kbps). NFC technology is based on the principle of magnetic field induction, which allows the NFC interface to interact with other NFC interfaces located on other mobile computing devices 104 or retrieving information from tags with embedded RFID identification schemes. The NFC interface can provide activation and/or acceleration of data transfer from one personal computer 104 to another personal computer 104 with an extremely close range (approximately 1½ inches).

The memory 206 associated with system 104, 108 may be, for example, one or more buffers, flash memory, or non-volatile memory, including random access memory RAM. The personal computer 104, 108 may also include non-volatile memory. The non-volatile memory may be any suitable storage medium, such as a hard disk or non-volatile memory, flash memory in particular. The memory 206 may include at least one database 207, which will be described in more detail below; this database is connected to the processor device 208 of personal computer and/or the mobile computing device 104, 108. In an embodiment of the invention, where the database 207 is considered to be at least a part of the memory 206 of the personal computer 104, 108, such a communication link may be a hard-wired connection. In an embodiment of the invention, where the remote database 106 is considered to be the database 207, accessible via, for example, long-distance networks, such as Internet/WAN/LAN 100, such a communication link can be established through the network interface 204 with the mobile computing device 104, 108. The term “database” is broadly used for an ordered set of data that is stored in non-volatile memory and is available for a data processing device that uses the set of data to solve tasks determined by a computer.

An example of a data processing device 208 is a central processing unit, a microcontroller, or a microprocessor system with a “general purpose” microprocessor or a “special purpose” microprocessor.

The processing device 208 executes the code stored in the memory 206 to execute operations/commands from the personal computer and/or a mobile computing device 104, 108. The data processing device 208 may provide processing capability for operating system management, running various applications, and processing data for implementing one or more of the methods described herein.

A display 210 displays information for the user, including operating status, time, contact information, various menus, application icons, pop-up menus, and so on. A display 210 may be used to display various images, text, graphics or videos, in particular, photographs, mobile television content, web pages, and mobile application interfaces of the user. One example of the configuration of a display 210 is displaying a subject cardiointervalogram as is described below. A display 210 may be any type of suitable display, including a liquid crystal display, a plasma display, a LED display, etc.

A personal computer and a mobile computing device 104, 108 include audio input/output components 212, such as a microphone for receiving audio signals from the user and/or a speaker for playing audio signals, such as audio recordings associated with the user's speech and/or any sounds, etc. The personal computer and a mobile computing device 104, 108 may also include an audio port for peripheral audio input and output components, such as a headset, peripheral speakers, or microphones.

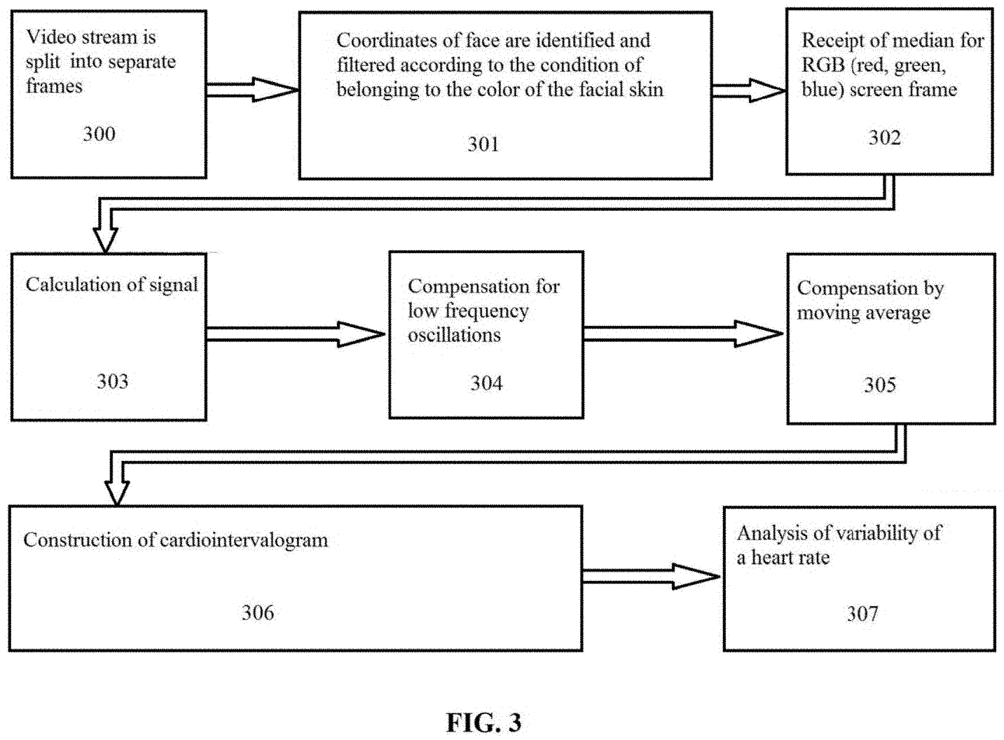

FIGS. 1-2 and FIGS. 4-9 will be described in connection with the process flow diagram shown in FIG. 3. Although FIG. 3 shows the specific order of the steps in the process, the order may be changed and may not correspond to the order used in some embodiments of the invention. In addition, two or more blocks depicted sequentially can be executed simultaneously or with partial coincidence in some embodiments. To make the description shorter, some steps can also be omitted in FIG. 3. In certain embodiments of the invention, some or all of the process steps shown in FIG. 3 can be combined into one process.

The process example shown in FIG. 3 begins at step 300 and continues to step 307, where a subject instructs the system to begin to determine heart rate variability parameters. The subject's command to start is received through the user interface 202 of the personal computer and/or mobile computing device 104. A “Start” or “Launch” engagement command can be created for a subject to confirm that they are ready to initiate the process of determining the cardiointervalogram parameters. A subject does not change their body position and their distance relative to a camera 200 (FIG. 2) for a certain period of time sufficient to determine the parameters of the heart rhythm, which is referred as the “determination period”. It should be clear to those skilled in the technical field that there are a number of other ways for the subject to engage a “start” command or another launch command, in particular, a voice signal recognition command, or other methods/structures for entering a user message into the personal computer and/or mobile computing device 104. In accordance with an additional option, after the subject sends a command to start the cardiointervalogram determination period, a display 210 and/or an audio input/output 212 may replay a countdown or otherwise prepare the subject to take the correct position in front of a camera before the start of the “determination period”. As for the exemplary embodiment, a display 210 counts down from 5 to give the subject 5 seconds to take the correct position. It is important that the subject is properly positioned relative to a camera 200, and a camera is able to capture the subject's image/video during the determination period, particularly subject's facial area. Tests have shown that serious interference that distorts the subject's face image, such as thick glasses, heavy makeup, covering of a face, extremely bright or dark lighting will adversely affect the accuracy of the measurements. Therefore, it is recommended to remove glasses, a heavy makeup, and have a reasonable amount of light (not too dark, not too bright), not chew and talk only when asked during the engagement, to breathe normally.

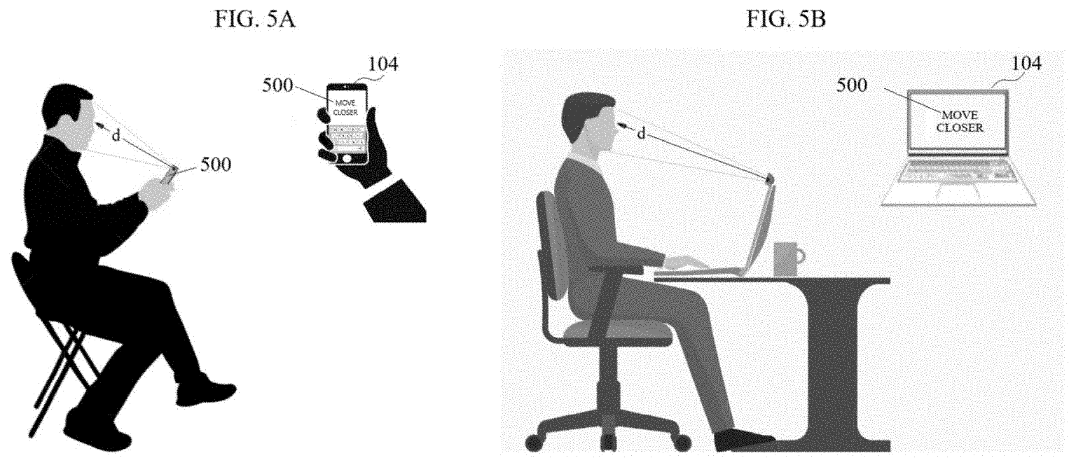

During the determination period, the subject must sit still at exactly the same distance from a camera 200. In one embodiment, the personal computer and/or mobile computing device 104 may prompt the subject to be located opposite a camera 200 within one separation distance d, as shown in FIG. 5 (A and B). The personal computer and or a mobile computing device 104 may advise the subject, for example, by means of a visual message 500, to “move closer,” “move further,” “stay in the same position,” or generate other similar messages that are displayed on a display 210 and/or reproduced as an audio signal using the audio input/output device 212 of a personal computer and/or mobile computing device 104 to ensure that the subject remains at a predetermined separation distance optimal for a camera 200 to record images/videos for subsequent processing.

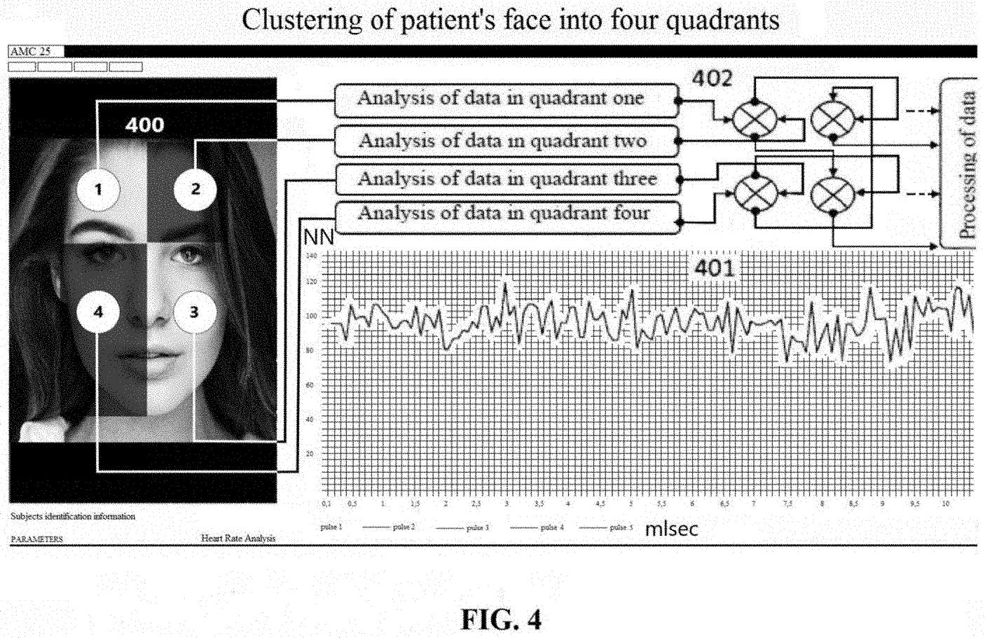

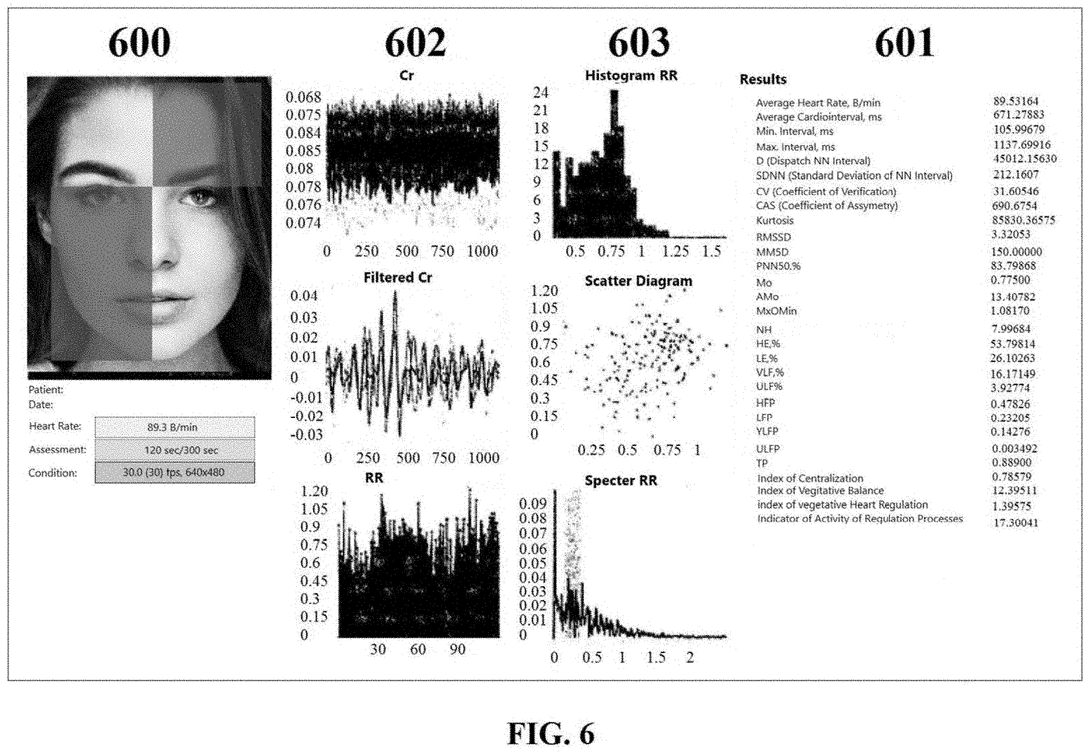

In order to visualize the stages of conversion of received graphic information via a camera 200 into data demonstrating parameters of vital functions of a human body, including analysis of variability of a heart rate 307 used by the claimed invention, the stages of this process are presented in the block diagram in FIG. 3. At step 300, the video stream is split into separate frames; at step 301 in the processor 208 (in FIG. 2), each pixel of the obtained image is filtered based on a condition of color scheme of the facial tissue studied, and the system determines coordinates of the boundaries of the areas to be studied; at step 302, the processor 208 calculates the average value for changes based on the applied RGB color model using clustering parameters (FIG. 4) for each frame; at step 303, the parameters of dynamic changes in the video stream are calculated based on the significance of the parameters of the applied color model to solve a diagnostic task; FIG. 6 shows an example of the calculation for the Cr component video signal to assess the frequency of external respiration and the parameters of second-order waves associated with this function; at stages 304 and 305, the system filters noise and compensates for low-frequency oscillations associated with the movement of the head; at steps 306-307, the processor 208 calculates the basic parameters of heart rate variability and builds a cardiointervalogram easy to understand and evaluate by a medical practitioner using the claimed telemedical system.

One of the important distinctive features of the claimed invention is the clustering of the parameters of the image of a face obtained from a camera 200 being a part of personal computer and/or mobile computing device 104 by the processor 208 using at least two projections; FIG. 4 shows the clustering of the subject's face into four quadrants 400, while the data processing is based on the mandatory assessment of the relationship between asymmetric changes in the color of the skin 402 and analysis of data on the optical density of tissues, plethysmography and oxygenation, as well as constructing a firm cardiointervalogram 401.

In order to record reliable graphic information that allows to determine and monitor data and parameters of vital functions of the human body, let us again review FIG. 5A, where a subject utilizes a mobile computing device with a built-in digital camera 200, holding it both hands at a focal distance—“d” Similarly, on FIG. 5B, a subject sits in front of a personal computer 104 with an integrated web camera 200 with mandatory coverage of the subject's face of least 70% looking at a display 210. The claimed invention provides for the possibility of recording data in any convenient for a subject location.

In order to ensure the visibility of the heart rate variability data obtained after conversion, FIG. 6 shows subject data 600 including statistical 601, geometric 602, and spectral 603 assessment data; during and upon completion this data is transferred to a medical practitioner in live feed for analysis and providing medical evaluation of received data utilizing the Internet/WAN/LAN Network 100 (in FIG. 1). The claimed invention provides for telemedical consultations by a medical practitioner, while ensuring the safety of the use of databases 207 (FIG. 2) and the accumulation of a large amount of subjects' personal data on the server 106 of the service provider; in addition, received new data is compared with the previously recorded data.

In order to use diagnostic methods and practices of interviewing and assessment of subject's audible data, the claimed invention provides for the possibility of synchronized input/output 212 of audio signals.

The algorithm for synchronized control of the asymmetry of oscillations of human physiological slow waves and emotional motor manifestations in the invention can be used by the subject and/or consultant for psychological testing and assessment of psychosomatic conditions, including detection of intoxication, stress and emotional overload.

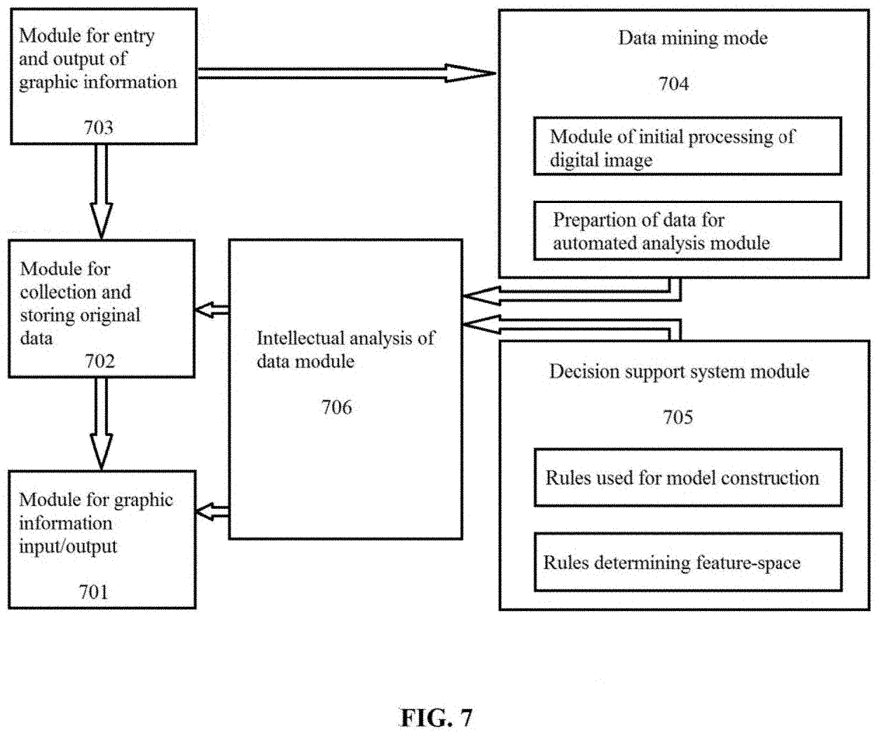

FIG. 7 shows a block diagram of a set of logical structures that implement various stages of the process, corresponding to the application of the claimed invention. The block diagram illustrates ability to transmit data from the graphic information input and output module 701 of the personal computer or mobile computing device 104, transmit data to the module for collecting and storing the original data 702 included in the system memory 206 (FIG. 2) for long-term storage and the module for collecting and processing information 703 incoming to system 208 for the purpose of transmitting data for processing in the data mining module 704 and further comparing and analyzing data from the decision support system module 705. After comparing and analyzing the data, the effects can be transmitted using the output module 706 to the server 106 or via the Internet/WAN/LAN 100 network (FIG. 1).

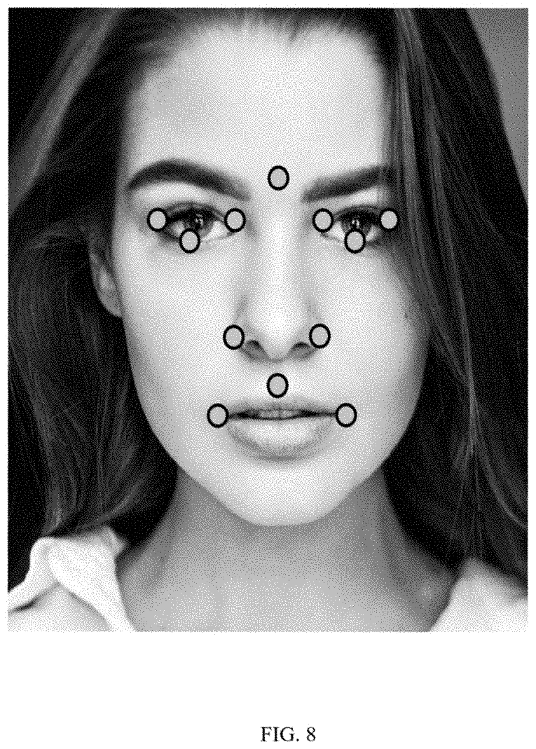

The processor 208 algorithm provides a coordinate capture of not less than 12 control points from the projection of the main sensory and motor cranial nerves (FIG. 8), as well as their vector variation. During the consultations, the algorithm allows to detect emotionally conscious and unconscious micro mimic facial expressions of a person, which can be used in the detection system to assess the reliability of perceived and transmitted information, as well as when using test tasks.

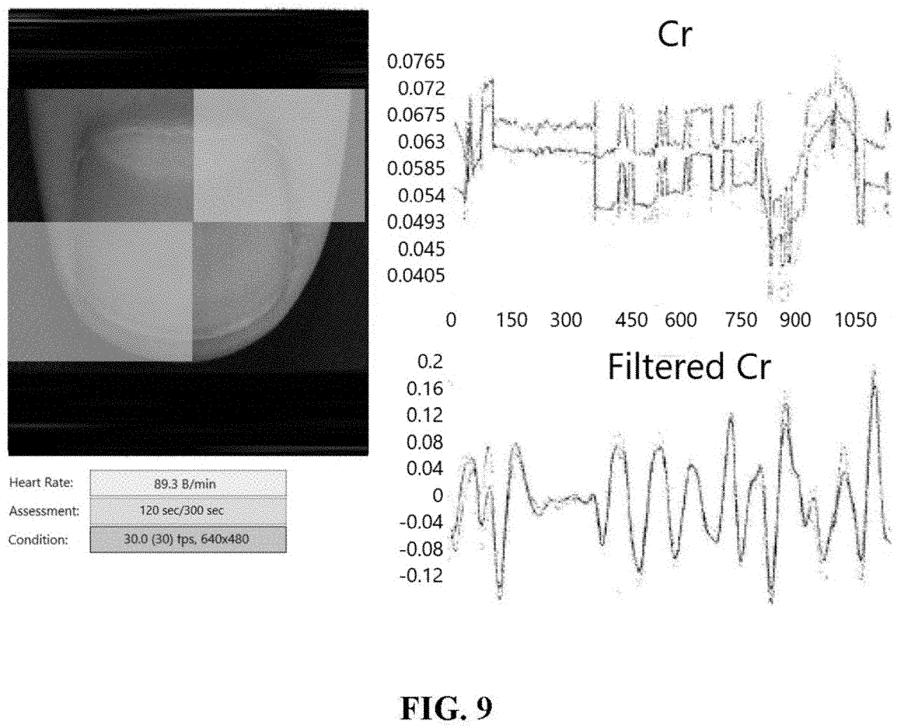

In accordance with the illustration of the diagnostic methods provided by claimed invention, monitoring of the parameters of the amplitude of oscillations of slow physiological waves from subject's different skin surfaces is supported. To this end, the system provides the ability to determine the measurement area ranging from 2 to 400 square centimeters; FIG. 9 shows an example of the registration of parameters of plethysmography from the nail phalanx of a person's finger.

Thus, the claimed invention allows remotely without using any tactile devices to detect on early stages and monitor the risks of functional disorders, an effectiveness of preventive measures according to the results of the analysis of the asymmetry of microcirculation of blood through the facial vessels and facial muscle contractions.

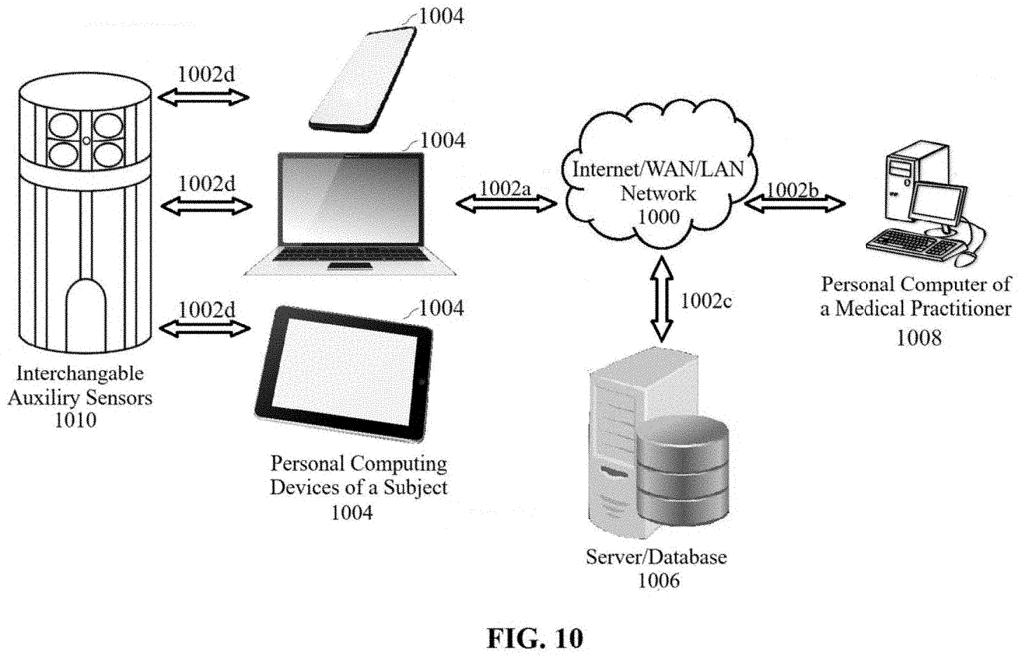

Another embodiment depicted in FIGS. 10-14 provides for another new and effective telemetry method and system for objectively examining the state of the human body condition during medical examination using remote monitoring by a medical practitioner as well as automated pre-scheduled medical assessment with or without a presence of a medical practitioner. It should be understood that the presented embodiments of the invention are merely examples that may be altered in various forms.

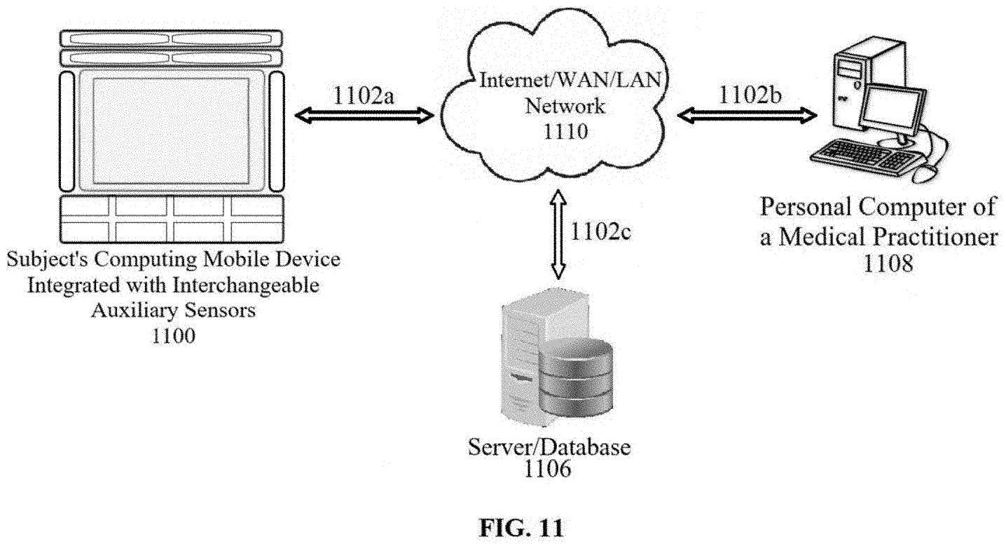

With reference to FIGS. 10-11, embodiments of the claimed invention are depicted and include an interchangeable auxiliary hardware system 1010 containing corresponding sensors and emitter devices in accordance with the projected use as described herein, including an optional camera and input/output audio device. The interchangeable hardware system 1010 may be in the form of single device, multiple devices, and may be separated from other electrical components or integrated within (as depicted in FIG. 10). Similar to FIG. 1, the interchangeable hardware system 1010 may be communicatively coupled with subject's personal computer and/or mobile computing device via one or more wired or wireless communication connections 1002d or may be integrally coupled with a mobile computing device 1100 as demonstrated on FIG. 11. The interchangeable hardware system may be coupled with optional medical electronic devices depending on a necessity of a subject's conditions and medical practitioner's recommendations that are outside this invention, e.g., a pulse oximeter or a heart monitor communicatively coupled thereto using, for example, one of the multiple types of possible connections 1002a-d, wherein the 1002a-d connections, which are the medium for communication between various system and computers interconnected in network 1000. Connections 1002a-d may be wired or wireless. Examples of wired connections are cable, telephone line, and fiber optic cable. Examples of wireless connections are Internet, radio frequency (RF), WiFi, Bluetooth, Infrared (IR), cellular mobile connections, etc. Many other wired and wireless connections are known in this technical field and can be used concurrently with the claimed invention.

In this example, an Internet/WAN/LAN Network 1000 includes an electronic system, such as subject's personal computer and/or a mobile computing device 1004, or integrated hardware system integrally coupled with a mobile computing device 1100 (FIG. 11), and interchangeable auxiliary hardware system 1010, a server/database 1006, and a personal computer of a medical practitioner 1008 with downloaded and/or preinstalled additional software for telemetric diagnostics of physiological and mental state of a subject, including assessment of a drug and alcohol intoxication.

Subject's personal computer and/or a mobile computing device 1004 may be used to execute programming commands contained in software that can be obtained from server 1006 via Internet/WAN/LAN Network 1000. In other embodiments of the invention, a personal computer of a medical practitioner 1008 may execute programming commands received from the server 1006 over the Internet/WAN/LAN Network 1000. In other embodiments, the software is a web application, desktop software, or a mobile application. In one embodiment, the WAN is the Internet. The Internet is a worldwide collection of networks and gateways that use the TCP/IP protocol suite to communicate with each other. The Internet is based on a unifying backbone consisting of high-speed data lines between the main nodes or host computers represented by thousands of commercial, government, educational, and other computer systems that route data and messages. Of course, network 1000 may also be implemented as many different types of networks, such as, for example, an intranet, a LAN, or a cellular network. FIGS. 10-11 are considered as an example rather than a structural limitation for the claimed invention.

The server 1006 can be considered as a computer that controls access to the centralized resource or database. In some embodiments of the invention, users of the personal mobile computing device 1004 may request the software, which is an example of the use of the claimed invention. The server 1006 can receive, process, and execute requests by transmitting the software to the subject's personal computer and/or a mobile computing device 1004 via Internet/WAN/LAN Network 1000. In other embodiments, the personal computer of a medical practitioner 1008 may request the software, and the server 1006 may receive, process, and execute the request by transmitting the software to the personal computer of a medical practitioner 1008 via Internet/WAN/LAN Network 1000.

Still referring to FIGS. 10-11, it can be noted that embodiments of the present invention are shown as block diagrams illustrating an exemplary network of a data processing system in which the present invention may be implemented. Said figures depict some of the advantages of the present invention, but, as will be described below, the invention can be presented in several forms, sizes, combinations of properties and elements, and with different numbers of components and their functions. The first example of a multifunctional corporate telemedicine Internet platform that provides the organization of medical examinations and access to large databases and knowledge, combining information and computing components, contains an intellectual analysis system, an interactive consultation system and a decision support system. Internet/WAN/LAN Network 1000 and Internet/WAN/LAN Network 1100, as shown in FIGS. 10-11 includes connections 1002a-d, which are a medium used to provide communication links between client and server end, various devices, and computers interconnected in Internet/WAN/LAN Network 1000 and Internet/WAN/LAN Network 1100. Connections 1002a-d can be wired or wireless connections.

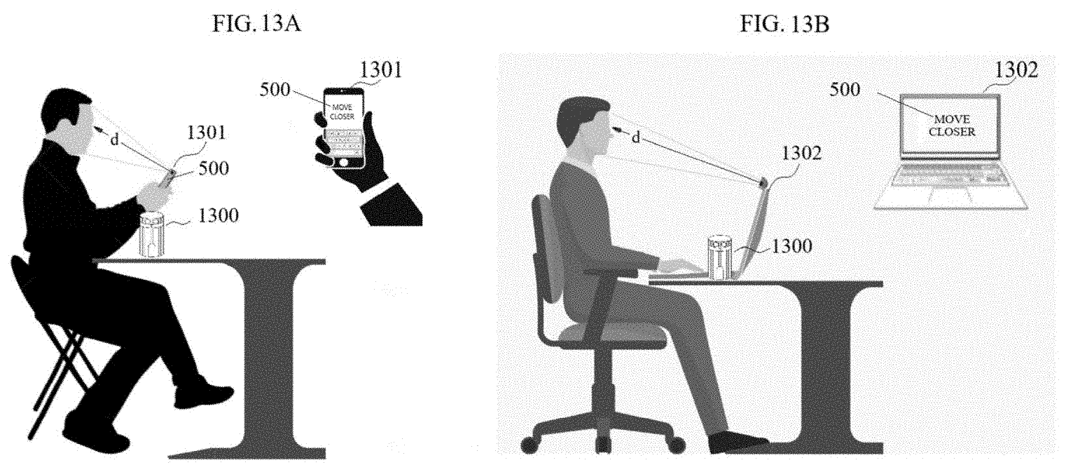

With reference to FIGS. 10-14, the Internet/WAN/LAN Network 1000 and Internet/WAN/LAN Network 1100 require a subject on FIG. 13 (A and B) to be in close proximity to subject's personal computer 1302 and/or a mobile computing device 1301 connected with an interchangeable auxiliary hardware system 1300, or to integrated hardware system integrally coupled with a mobile computing device 1100 as demonstrated on FIG. 11, a medical practitioner to be at their personal computer 1008, and a server 1006 all communicatively coupled together over a network. In case of an automated pre-scheduled medical assessment, a medical practitioner is not required to be at their computer. The software and interchangeable auxiliary hardware system for telemetric control of the physiological and mental state of an subject can be used to execute programming commands contained in the software, which can be obtained from the server 1006 over the Internet/WAN/LAN Network 1000. In one embodiment, the Internet/WAN/LAN Network 1000 is a wide area network (WAN) and is the Internet. Of course, Internet/WAN/LAN Network 1000 can also be implemented as many different types of networks, such as, for example, a local area network (LAN), or cellular network.

The server 1006 can be considered as a computer that controls access to the centralized resource or database. In some embodiments, users of both personal computer and mobile computing devices 1004 may request a software application exemplifying the use of the present invention. The server 1006 can receive, process, and execute the request by transmitting the software application to the personal computer and mobile computing devices 1004 via Internet/WAN/LAN Network 1000, issue recommendations via the server 1006 and the Internet/WAN/LAN Network directly.

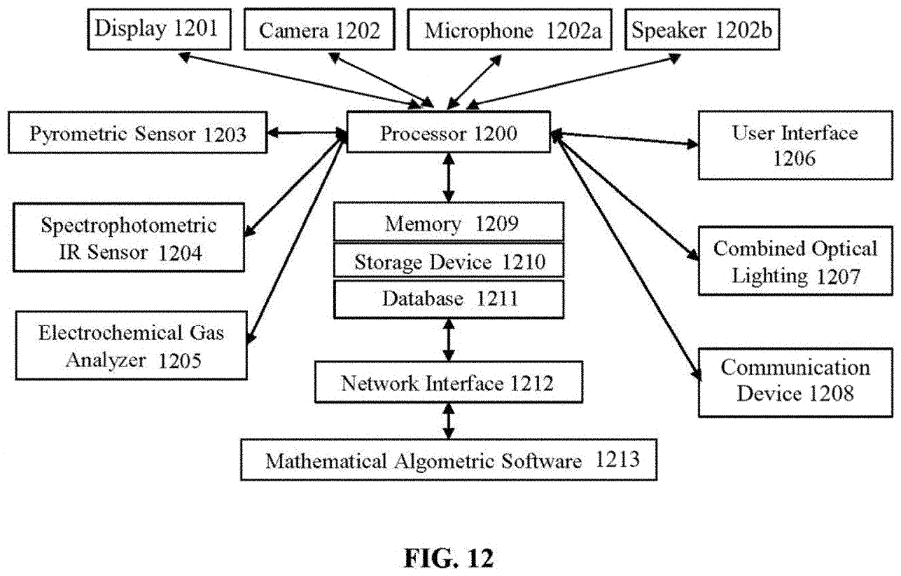

Referring now to FIG. 12, in the above example, the software and interchangeable auxiliary hardware system may include a processor 1200 included in a personal computer and/or mobile computing device equipped with a display 1201, camera 1202 connected to a microphone 1202a and a speaker 1202b, pyrometric sensor 1203, spectrophotometric IR sensor 1204, electrochemical gas analyzer 1205, user's input interface 1206, combined optical lighting 1207, communication device 1208, memory 1209, storage device 1210, database 1211, network interface 1212 and mathematical algorithmic software 1213.

Mathematical algorithmic software 1213 may carry out switching connections of the processor 1200 with devices and sensors that provide biomonitoring of the subject, such as the pyrometric infrared sensor 1203, which monitors the surface temperature of the body, spectrophotometric infrared sensors 1204 and an electrochemical gas analyzer 1205 extremely sensitive to ethanol for monitoring the gas environment as well as combined optical lighting 1207 and a camera 1202.

The camera 1202 captures still as well as video images. The camera 1202 is digital with automatic focal length detection. The camera 1202 is configured so that images can be stored in memory 1209 and processed by a mathematical algorithmic software 1213.

The camera 1202 is operably connected to the microphone 1202a for the purpose of synchronizing the recording of audio and video images. The camera 1202 is also configured to capture images having a pixel resolution of at least 640×480 pixels to provide a high resolution for interpreting and analyzing images in accordance with the techniques described herein and generally known in the art. Cameras of lower quality may not be able to provide images at the proper resolution. The user input interface 1206 operates to provide a method for inputting data from a user into a personal computer and/or mobile computing device. A digital camera may be a part of a personal computing device or may be integrated as part of an interchangeable auxiliary hardware system.

The network interface 1212 may include one or more network interface cards (NICs) or a network controller. In some embodiments, the network interface 1212 may include a personal area network (PAN) interface. The PAN interface can provide the ability for the processor 1200 to connect to a network using a short-range communications protocol such as Bluetooth. The PAN interface allows a processor 1200 to establish a wireless connection with another processor through a peer-to-peer connection.

The network interface 1212 may also include a local area network (LAN) interface. The LAN interface can be, for example, a wireless LAN interface such as a Wi-Fi network. The range of the LAN interface can usually exceed the range available through the PAN interface. In most cases, the connection between two electronic devices over a LAN interface may involve a connection through a network router or other intermediate device.

In addition, the network interfaces 1211 may include WAN connectivity over a wide area network (WAN) interface. The WAN interface can provide connectivity to, for example, a cellular mobile network. The WAN interface may include communication setups such as an antenna coupled to a radio circuit having a transceiver for transmitting and receiving radio signals through the antenna. The radio circuit can be configured to operate in a mobile network, including, but not limited to, global systems for mobile communications (GSM), code division multiple access (CDMA), wideband CDMA (WCDMA), and the like.

The processor 1200 may also include a near field communication (NFC) interface. The NFC interface can provide an extremely close communication radius at relatively low data rates (e.g., 424 kbps). The NFC interface can function by magnetic induction, allowing the NFC interface to interact with other NFC interfaces located on other devices, or extract information from tags that have radio frequency identification (RFID) schemes. The NFC interface can enable and/or accelerate data transfer from one personal computer to another personal computer with an extremely close range (e.g., 1½ inches).

The memory 1209 associated with devices utilized over the network can be, for example, one or more buffer, flash memory, or non-volatile memory such as random-access memory (RAM). Any computing device may also include nonvolatile memory. The non-volatile storage device can be any suitable storage medium such as a hard disk or non-volatile memory such as flash memory. The Memory 1209 may include at least one database 1211 connected to storage device 1210 of device. In an embodiment in which database 1211 is considered at least part of memory 1210 of personal computer or a mobile computing device, such communication may be hard-wired. mounted conductive connection. In an embodiment of the invention, in which the database 1211 is considered to be a remote database accessible via, for example, a long-distance network such as Internet/WAN/LAN Network, such communication may be established via the network interface 1212 of the device. The term database in a broad sense used to denote an ordered set of data that is stored in non-volatile memory and is available to a data processing device using the data set to solve computer-defined tasks.

A processor 1200 can be, for example, a central processing unit (CPU), microcontroller, or microprocessor device including a general-purpose microprocessor or a special purpose microprocessor. Processor 1200 executes code stored in memory 1209 to perform operations/commands of a personal computer and/or mobile computing device 1004. The processor 1200 may provide processing capabilities for operating system control, launching various applications, and processing data for one or more of methods described here.

The personal computer and/or mobile computing device monitor/display 1201 displays information to the user such as operating status, time, contact information, various menus, application icons, pull-down menus, and the like. Display 1201 can be used to present various images, text, graphics, or videos to a user, such as photographs, mobile TV content, web pages, and mobile application interfaces. In a specific case, display 1201 may be configured to display the user's cardiointervalogram, as described in the claimed invention. Display 1201 can be any type of suitable display such as a liquid crystal display (LCD), plasma display, light-emitting diode (LED) display, and the like.

The computing device may also include audio input and output structures such as a microphone 1202a for receiving audio signals from a user and a speaker 1202b for outputting audio signals such as audio recordings associated with the subject's speech and/or any sounds, movements, and etc. The personal computer and a mobile computing device may also include an audio port for connecting to peripheral audio input and output structures such as a headset, peripheral speakers, or microphones.

FIG. 13 (A and B) demonstrates an example of a process of conducting a medical examination using introduced software and interchangeable auxiliary hardware system 1300. The process begins with a subject consenting to initialize a medical examination to a medical practitioner using personal computer 1302 or a personal mobile computing device 1301 command.

Before undergoing the examination, a subject sits down on a chair in front of a computing device 1301, 1302 taking a comfortable position, straightens legs, hands are place in front on top of the table. It is not recommended to talk or make any movement during the examination unless asked. The subject should not place himself/herself further than 3 feet 1 m) away from the camera and not more than 2 feet 30 cm) from interchangeable auxiliary hardware 1300, rotate their head or twist their body. Must sit calm. Tests have shown that serious interference that distorts the subject's face image, such as thick glasses, heavy makeup, covering of a face, extremely bright or dark lighting will adversely affect the accuracy of the measurements. Therefore, it is recommended to remove glasses, a heavy makeup, and have a reasonable amount of light (not too dark, not too bright), breathe normally. Once the medical examination starts, a medical practitioner will instruct for further actions.

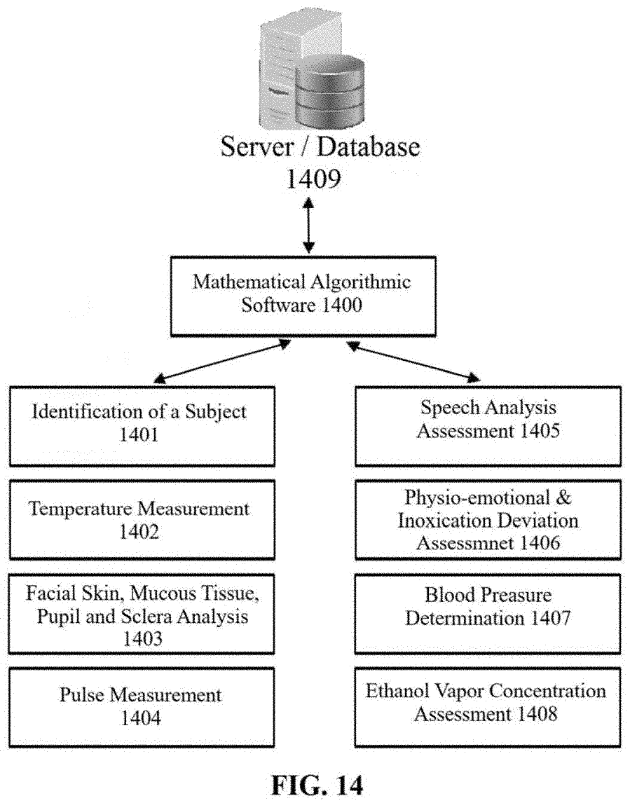

FIG. 14 shows a block diagram demonstrating intercommunication between and operation of a software and interchangeable auxiliary hardware system sensors and its main functions that allow to conduct a thorough telemedical examination of a subject, including clinical signs of drug or alcohol intoxication remotely. The software and interchangeable auxiliary hardware system is supported by a mathematical algorithmic software 1400. The system provides data retrieval and transmission of staged data transfer using server 1409 connected with subject's computing device and medical practitioner's personal computer via Internet/WAN/LAN Network 1409, as described at FIGS. 10 and 11. The first stage of operation includes steps that provide an identification of the subject and comparison with the database of stored information about the subject. Once the biometric data match established, the server 1409 sends a confirmation of subject's identity to a medical practitioner to allow to start the remote medical examination. During the second stage of the process, a temperature measurement 1402 is taken and transmitted to a personal computer of a medical practitioner.

The third stage engages operation of computer vision technologies 1403, which allow to identify and analyze the color gradient and surface area assessment of the facial skin tissues, including visible mucous membranes, conduct a pupil and sclera visual analysis. As described previously in the art, a pulse 1404 is measured and sent to the server for comparison with reference to personal values; the result and data demonstrating deviation from “norm” is sent to a medical practitioner for evaluation. The speech analysis assessment 1405 is carried based on medical practitioner's decision utilizing a provide an automated questionnaire session from a mathematical algorithmic software 1400. During the examination, a medical practitioner determines an average execution time of the task and the number of errors that a subject had made. The execution of psycho-emotional and intoxication assessment 1406 is carried out by utilization of computer vision and artificial intelligence algorithms being a part of the mathematical algorithmic software 1400, allowing to monitor facial mucus tissues discoloration, pupil tracking and sclera analysis, monitoring of which lasts during the entire medical examination from the moment subject's identity confirmation, while receiving live feed data, including alcohol intoxication assessment by utilizing interchangeable auxiliary hardware sensor of ethanol vapor concentration 1408. Based on the results of evaluation of the parameters of physiological data by means of computer vision technology a mathematical algorithmic software 1400 provides measurement data of a blood pressure 1407 for a medical practitioner to conclude the telemedical remote examination.

In another embodiment, at the end of the medical examination, a mathematical algorithmic software 1400 may provide its recommendations based on the conventional treatment methods to a medical practitioner to be taken into consideration when providing diagnosis and recommendations.

The reliability of data obtained using the telemetric monitoring system and the method of obtaining parameters of vital human functions were verified by conducting a trial, where the correlation between the physiological parameters of trial subjects recorded using the telemetric monitoring system and standard diagnostic methods were based on sixty subjects taking part in the trials: 30 trial subjects were considered conditionally healthy, and 30 trial subjects received a confirmed diagnosis of somatoform autonomic dysfunction. All trial subjects were given mobile computing devices in from of a tablet, equipped with a webcam, microphone, and Internet access. The tablets also had a pre-installed software program of the telemetric monitoring system of parameters of vital human functions with Internet access and access to the server of the telemetric monitoring system. In order to confirm the reliability of data obtained using the telemetry system, the following standard diagnostic system and methods were used in the trials (1) Electrocardiograph (ECG), (2) Doppler ultrasound (USDG), (3) Tissue oximeter: OxiplexTS, ISS Inc., USA, (4) Magnetic cardio-encephalograph (MCEG), and (5) Photoelectric pulse oximeter.

Parameters of chronobiological oscillatory components and constants of vital functional parameters of self-regulation of organs and body systems were subject to assessment: (1) Heart rate, variational pulsometry and oximetry, (2) Slow heart rate waves: ULF, VLF, LF, and (3) Respiratory waves.

As the result of this research, data obtained confirmed the high informativity of the created system and the developed methods for diagnosing the functional state and allowed to create algorithms for correlation with standard clinical methods. The goal of the second stage of the research was to assess the possibility of using telemedical software to remotely assist subject to correct the functional state of those diagnosed with somatoform autonomic dysfunction. Each trial subject had their heart rate variability parameters recorded in a sitting position. The data obtained were evaluated by a cardiologist. After that, the Stange-Hench tests were carried out, and the average inspiration breath hold for all the trial subjects did not exceed 30 seconds.

In order to increase adaptive capacity, trial subjects were asked to conduct autogenic respiratory training for 9 minutes with a motivated control of their own pulse and respiratory waves on the graphical display of the telemetric control system. At 60 minutes after the training, Stange-Hench test results demonstrated an increase in average breath hold of trial subjects up to 40 seconds.

What is claimed is:

1. A software and interchangeable auxiliary hardware system for telemetric control of the physiological and mental state of a subject comprising of:
an electronic computing device of a subject operably connectable with interchangeable auxiliary sensors, having an integrated camera, and having an audio input and an audio output, the electronic computing device:
operably configured to measure spectroscopic changes associated with the subject, parameters of color changes in facial skin and the mucous membranes tissues of the subject, conduct pupil assessment and a sclera discoloration analysis, measure surface temperature of skin of the subject, composition of exhaled air from the subject, and sensorimotor reaction of the subject, motor activity of the subject; optical lighting operably configured to assess skin color and perfusion of blood vessels of the subject's face; and
communicatively coupled to a processor operably configured, through programed software instructions, to process the registered data received from the measurements obtained by the interchangeable auxiliary sensors to be determined an overall health condition of the subject, including clinical signs of drug and alcohol intoxication that may impede general wellbeing of the subject, and to generate recommendations for a medical practitioner to consider via a wireless network with an electronic computing device of a medical practitioner communicatively coupled thereto.

2. The software and interchangeable auxiliary hardware system for telemetric control of the physiological and mental state of a subject according to claim 1, wherein the computing device of the subject further comprises of:
a graphic information input/output device operably configured to control at least one of the registered parameters.

3. The software and interchangeable auxiliary hardware system for telemetric control of the physiological and mental state, including states of intoxication of a subject according to claim 2, wherein the computing device of the subject further comprises:
a graphic information input device operably configured to control a color gradient of image elements of the subject's skin, in dynamics with a resolution of at least 30 frames per second.

4. The software and interchangeable auxiliary hardware system for telemetric control of the physiological and mental state of a subject according to claim 3, wherein the computing device of the subject further comprises:
a user interface operably configured to view registered parameters including hemodynamic oscillations of the blood filling of the vessels of the subject's face.

5. The software and interchangeable auxiliary hardware system for telemetric control of the physiological and mental state of a subject according to claim 4, further comprising:
a pyrometric infrared sensor operably configured to obtain a registered parameter that includes the temperature of the surface of the subject's facial skin and detecting correlation and blood perfusion oscillation registration parameters and surface temperature gradient.

6. The software and interchangeable auxiliary hardware system for telemetric control of the physiological and mental state of a subject according to claim 5, wherein multiple sensors are operably configured to measure pscychoemotional and mental state of the subject.

7. The software and interchangeable auxiliary hardware system for telemetric control of the physiological and mental state of a subject according to claim 5, wherein multiple sensors are operably configured to measure clinical signs of alcohol and drug intoxication.

8. The software and interchangeable auxiliary hardware system for telemetric control of the physiological and mental state of a subject according to claim 5, further comprising:
an audio information input device operably configured to register voice data and phoniatric changes of the subject during an interrogation process.
